(19) United States
(12) Patent Application Publication
(10) Pub. No.: US 2025/0072862 A1
(43) Pub. Date: Mar. 6, 2025

(54) CIRCUITRY AND METHOD

(71) Applicant: Sony Group Corporation, Tokyo (JP)

(72) Inventor: Justinas MISEIKIS, Stuttgart (DE)

(73) Assignee: Sony Group Corporation, Tokyo (JP)

(21) Appl. No.: 18/817,334

(22) Filed: Aug. 28, 2024

(30) Foreign Application Priority Data
Sep. 5, 2023 (EP) 23195509.7

(51) Int. Cl. A61B 8/08; A61B 8/00

(52) U.S. Cl. CPC A61B 8/0866; A61B 8/4245; A61B 8/463; A61B 8/483

(57) ABSTRACT

The disclosure pertains to a circuity for generating a visualization of a fetus, wherein the circuitry is configured to: obtain ultrasound data representing a fetus in a uterus of its mother; establish, based on the ultrasound data, a data model of the fetus; and generate, based on the data model, a visualization of the fetus using a generative artificial intelligence.

### CROSS-REFERENCE TO RELATED APPLICATION

The present application claims priority to European Patent Application No. 23195509.7, filed on Sep. 5, 2023, the content of which is incorporated herein by reference in its entirety.

### TECHNICAL FIELD

The present disclosure generally pertains to a circuitry and a method, in particular, to a circuitry and a method for generating a visualization of a fetus.

### TECHNICAL BACKGROUND

It is generally known to perform ultrasound imaging of a fetus. Ultrasound imaging is in some instances based on emitting an ultrasound signal into a belly of a mother while the fetus is in a uterus of the mother, and detecting a reflection of the ultrasound signal. Based on an intensity of the reflection as well as on a delay between emitting the ultrasound signal and detecting the reflection, an image of the fetus is reconstructed.

Although there exist techniques for ultrasound imaging of a fetus, it is generally desirable to provide an improved circuitry and method for generating a visualization of a fetus.

### SUMMARY

According to a first aspect, the disclosure provides a circuity for generating a visualization of a fetus, wherein the circuitry is configured to: obtain ultrasound data representing a fetus in a uterus of its mother; establish, based on the ultrasound data, a data model of the fetus; and generate, based on the data model, a visualization of the fetus using a generative artificial intelligence.

According to a second aspect, the disclosure provides a method for generating a visualization of a fetus, wherein the method includes: obtaining ultrasound data representing a fetus in a uterus of its mother; establishing, based on the ultrasound data, a data model of the fetus; and generating, based on the data model, a visualization of the fetus using a generative artificial intelligence.

Further aspects are set forth in the dependent claims, the drawings and the following description.

### BRIEF DESCRIPTION OF THE DRAWINGS

Embodiments are explained by way of example with respect to the accompanying drawings, in which:

FIG. 1 illustrates an embodiment of a circuitry;

FIG. 2 illustrates an embodiment of a method; and

FIG. 3 illustrates an embodiment of a general-purpose computer.

### DETAILED DESCRIPTION OF EMBODIMENTS

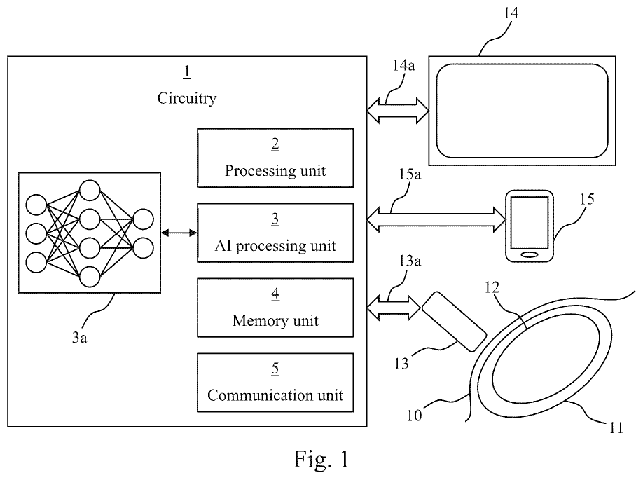

Before a detailed description of the embodiments under reference of FIG. 1 is given, general explanations are made.

As mentioned in the outset, it is generally known to perform ultrasound imaging of a fetus. Ultrasound imaging may be based on emitting an ultrasound signal into a belly of a mother while the fetus is in a uterus of the mother, and detecting a reflection of the ultrasound signal. Based on an intensity of the reflection as well as on a delay between emitting the ultrasound signal and detecting the reflection, an image of the fetus may be reconstructed.

For example, an amazing process for expecting parents during pregnancy may be getting an ultrasound check during a visit at a doctor (e.g., typically a gynecologist) and seeing a real-time image of the baby inside the womb. It may also be crucially important for the doctor to get a good scan, perform precise measurements and understand a size, a position and certain aspects of a growth of the baby to ensure healthy development, or take some countermeasures and treatments as early as possible if some issues are noticed.

However, in some instances, the ultrasound check typically provides a two-dimensional (2D) image, and good skills of positioning an ultrasound probe and reading the image are needed in order to obtain a correct image and understanding precisely of what is visible in the image.

For an unskilled person, such as typical expectant parents, it may be extremely difficult to make sense of different body parts, and especially position and size of the baby inside the womb, based on the image. Even though some three-dimensional (3D) reconstruction algorithms may already exist on medical ultrasound devices, such existing 3D reconstruction algorithms may still not provide a clear image and understanding of the state and pose (e.g., position, orientation) of the baby.

Consequently, some embodiments of the disclosure pertain to a circuity for generating a visualization of a fetus, wherein the circuitry is configured to: obtain ultrasound data representing a fetus in a uterus of its mother; establish, based on the ultrasound data, a data model of the fetus; and generate, based on the data model, a visualization of the fetus using a generative artificial intelligence.

The circuitry may be configured to execute instructions (e.g., software, firmware), and may include a programmed microprocessor (e.g., a central processing unit, CPU), an application-specific integrated circuit (ASIC), a field-programmable gate array (FPGA) or the like. The instructions may cause the circuitry to provide a functionality related to generating a visualization of a fetus.

The circuitry may include a memory that stores the instructions, the obtained ultrasound data, the data model of the fetus, the generated visualization, and/or (further) temporary values generated by the circuitry. The memory may include a flash memory, an electrically erasable programmable read-only memory (EEPROM), a dynamic random-access memory (DRAM), a static random-access memory (SRAM) or the like.

The circuitry may include a communication unit for obtaining the ultrasound data and/or for outputting the generated visualization of the fetus. The communication unit may be configured to communicate with another device via universal serial bus (USB), peripheral component interconnect (PCI), serial port (RS-232), parallel port (IEEE 1284), ethernet, wireless local area network (WLAN; e.g., Wi-Fi, IEEE 802.11), Bluetooth, high-definition multimedia interface (HDMI), DisplayPort, or the like.

The circuitry may include an artificial intelligence processing unit for executing the generative artificial intelligence. The artificial intelligence processing unit may include a graphics processing unit (GPU) and/or a tensor processing unit (TPU).

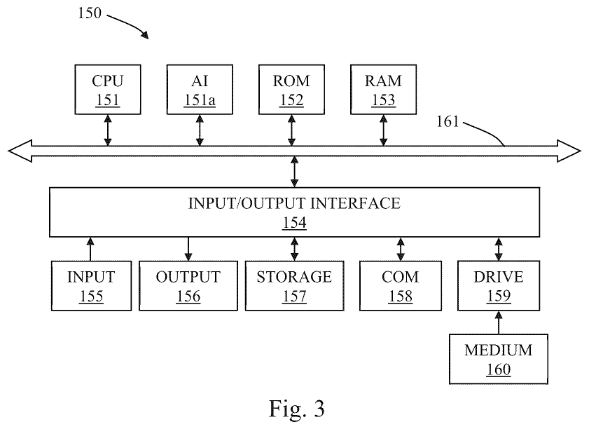

The circuitry may, for example, be realized as a general-purpose computer 150, as described with respect to FIG. 3.

The circuitry may obtain the ultrasound data from an ultrasound probe. The ultrasound probe may include an ultrasound source and an ultrasound sensor. The ultrasound source may be configured to emit a predefined ultrasound signal. The ultrasound signal may have a frequency in a range between 1 MHz and 40 MHz, without limiting the disclosure to this range. The ultrasound sensor may be configured to detect a reflection of the ultrasound signal including an intensity of the reflection and a delay between emitting the ultrasound signal by the ultrasound source and detecting the corresponding reflection by the ultrasound sensor. The ultrasound source and/or the ultrasound sensor may include a piezoelectric element for generating the ultrasound signal and/or for detecting the reflection, respectively, based on the piezoelectric effect. Other technologies for generating the ultrasound signal and/or for detecting the reflection may be possible as well.

When a user (e.g., a doctor who is performing an ultrasound examination of the fetus) positions the ultrasound probe at the belly of the mother, the ultrasound source may emit the ultrasound signal into the belly of the mother such that the ultrasound signal may reach the uterus of the mother and the fetus in the uterus. Different materials and/or types of tissue within the belly, the uterus and/or the fetus may absorb the ultrasound signal with different specific respective absorption coefficients and may attenuate the ultrasound signal accordingly. An inhomogeneity (e.g., an interface between different materials and/or types of tissue) within the belly, the uterus or the fetus (e.g., a surface of the fetus) may reflect the ultrasound signal. The ultrasound sensor may detect the reflection including its intensity and/or its delay after emission of the respective ultrasound signal.

The ultrasound source may emit a plurality of ultrasound pulses in the ultrasound signal and/or may emit a plurality of ultrasound signals, and the ultrasound sensor may detect the respective plurality of ultrasound pulses and/or ultrasound signals. For example, a user may move the ultrasound probe to different positions on the belly of the mother for obtaining ultrasound data of the fetus from different perspectives and/or for finding an optimum position from which a portion of interest of the fetus can be imaged. For example, the ultrasound source may emit subsequent ultrasound pulses into different directions and/or the ultrasound sensor may determine intensities and delays of respective reflections of ultrasound pulses from different directions, such that a field-of-view of the ultrasound probe (e.g., an area or solid angle covered by the ultrasound data) is increased.

The ultrasound data may indicate intensities of reflections of respective ultrasound signals and/or corresponding delays after emission of corresponding ultrasound signals. The ultrasound data may indicate respective directions from which the ultrasound sensor has received the reflections. The ultrasound data may represent an (two-dimensional (2D) or three-dimensional (3D)) image generated based on the intensities and/or delays and/or on the respective directions.

A description of further details of obtaining ultrasound data is omitted because ultrasound imaging generally is known in the art and the skilled person will find suitable techniques for obtaining the ultrasound data.

Accordingly, when a user places the ultrasound probe at the belly of the mother, the obtained ultrasound data may represent the fetus in the uterus of the mother.

The establishing of the data model of the fetus may include generating a three-dimensional (3D) representation of the fetus. For example, the data model may include a 3D spline representation of the fetus. For example, the data model may include a tessellated representation of the fetus, and may, e.g., indicate vertices and/or (triangular or polygonal) facets that represent the fetus. For example, the data model may include a 3D point cloud and/or a 3D grid that represent the fetus. The data model is not limited to these types, and the skilled person will find other suitable representations of the fetus in a data model.

The establishing of the data model may include identifying the fetus (or a part of the fetus) in the ultrasound data and omitting other structures (e.g., the uterus, bones or other tissue of the mother) represented by the ultrasound data, such that the data model can represent the fetus isolated from the other structures. Alternatively, the other structures may be included in the data model.

The generating of the visualization of the fetus may include inputting the data model into the generative artificial intelligence. The generative artificial intelligence (AI) may include or be based on an artificial neural network, e.g., a deep neural network, a convolutional neural network, a neural radiance field (NeRF), a variational autoencoder (VAE), a generative adversarial network (GAN), or the like. For example, the generative AI may use or may be configured similar to DALL-E, Midjourney, Stable Diffusion, Deep Dream Generator, StyleGAN, BigGAN, or the like.

The generative AI may be configured (e.g., trained) to generate a detailed image of the fetus based on an input that includes the data model and/or the ultrasound data. For example, the generative AI may remove noise or artefacts from the ultrasound data, which may still be present in the data model. The generative AI may generate the visualization of the fetus such that a posture of the fetus is visualized according to the ultrasound data. The visualization of the fetus may include a 2D or 3D image of the fetus. The generative AI may generate a 3D visualization of the fetus in which the fetus is shown in a projection as seen from a predefined perspective, and/or in which an attached shadow of the fetus is shown. The visualization of the fetus may be generated as a greyscale image and/or as a color image, wherein colors of the color image may correspond to true/realistic colors or to pseudo colors that may indicate respective predefined anatomic features (e.g., types of tissue, body parts of the fetus, deviation of an anatomic feature from a typical value, distinction between the fetus and the mother, or the like).

The circuitry may generate the visualization of the fetus in real time. For example, the ultrasound probe may generate ultrasound data based on respective ultrasound signals at different points in time (e.g., at a predefined rate; e.g., every second, every 100 ms, every 10 ms, every millisecond, every 0.2 milliseconds or the like, without limiting the disclosure to these values). The circuitry may obtain the respective latest ultrasound data, establish a data model of the fetus (e.g., update a data model established based on previous ultrasound data) based on the respective latest ultrasound data (or based on ultrasound data that are more recent than the last update of the data model) and generate a visualization of the fetus based on the respective latest (e.g., updated) data model (e.g., update a visualization generated based on a previous data model). For example, the circuitry may generate an updated visualization of the fetus at a predefined frame rate (e.g., 10 frames per second (fps), 30 fps, 50 fps, 100 fps or the like, without limiting the disclosure to these values).

The circuitry may write the generated visualization of the fetus to a storage (e.g., hard disk drive (HDD), solid-state drive (SSD), USB flash drive, Secure Digital (SD) card, or the like), upload the generated visualization to a server (e.g., via ethernet or Wi-Fi), and/or send the generated visualization to a display device for display (e.g., via Video Graphics Array (VGA), High-Definition Multimedia Interface (HDMI), DisplayPort, USB, Wi-Fi, Bluetooth, or the like). For example, the circuitry may send the visualization of the fetus to a screen in a surgery of a doctor (e.g., gynecologist) who performs an ultrasound check. For example, the circuitry may send the visualization of the fetus to a portable or wearable device (e.g., smartphone, head-mounted display, smartglasses, smartwatch, or the like) of a user (e.g., the doctor who performs the ultrasound check, the mother or the father of the fetus). The display device may display the visualization of the fetus.

Accordingly, some embodiments provide utilizing ultrasound scan information (e.g., the ultrasound data) combined with generative AI to give a precise insight about a state of a baby (e.g., the fetus) inside the womb (e.g., the uterus). If a well determined environment and structure of the fetus/baby in the womb is given that allows capturing certain details such as main body parts as well as developing organs such as brain, heart, spine, etc., a model (e.g., the data model) may be built up based on an initial scan using the ultrasound probe. A thorough ultrasound scan from multiple directions may be needed to capture sufficient details and establish the data model of the fetus/baby. Once enough details have been observed, the visualization of the fetus/baby may be created by adding a skeletal structure, current pose, size and pose in the womb.

By using the established data model, the fetus may be visualized in real-time, which may include adjusting and animating the visualization of the fetus, using real-time information of position changes, captured heart-beat and other aspects and vital signs of the fetus that can be determined based on the in the ultrasound data. The visualization may be displayed on a 2D screen, on a mobile device, or by an augmented reality (AR)/virtual reality (VR) device.

Thus, the circuitry may be configured to perform an AR visualization of the fetus/baby in the uterus/womb.

In some embodiments, the circuitry is further configured to: obtain image data of a portion of a belly of the mother; and determine a position of the fetus relative to the mother; and the generating of the visualization of the fetus includes generating the visualization according to the determined position.

The circuitry may determine the position of the fetus relative to the mother based on the ultrasound data and based on the image data. The circuitry may generate the visualization such that the visualization indicates the fetus in a posture and/or orientation that corresponds to a posture and/or orientation of the fetus in the uterus.

For example, the generating of the visualization of the fetus may include inserting into an image represented by the image data the visualization of the fetus according to the determined position of the fetus. The visualization may show the fetus within the belly of the mother, e.g., the fetus framed by the belly of the mother.

For example, the fetus may be “anchored” to a physical position of the mother. The position of the fetus may be observed in the ultrasound scan and anchored in relation to outside features of the belly indicated in the image data. This way, the created data model of the fetus and an exact position of the fetus inside the womb/uterus may be visualized in real-time and, thus may provide the user with an exact understanding on how the fetus is positioned, its body pose, movements, etc.

For example, the circuitry may perform a precise 3D reconstruction and high-quality creation of the data model of the fetus/baby including segmented body parts, which may be animated.

In some embodiments, the determining of the position of the fetus includes: identifying, in the ultrasound data, a structure of a body of the mother; and determining, in an image represented by the image data, a position that corresponds to the structure.

For example, the circuitry may identify, as a structure of the body of the mother in the ultrasound data, a bone (e.g., hip, vertebra, rib, etc.), a tissue, a muscle, an organ, a blood vessel, the uterus or the like. The circuitry may determine an orientation of the mother in the image data, e.g., based on a certain well-known position of a structure of an outside of the belly of the mother (such as belly-button, hips, chest, etc.). Based on the determined orientation of the mother in the image data, the circuitry may match a position of the structure identified in the ultrasound data with a position in the image data, e.g., according to a typical anatomic condition known in the art.

The circuitry may generate the visualization such that an orientation of the fetus in the visualization relative to the belly of the mother corresponds to an actual orientation of the fetus in the uterus relative to the belly of the mother. For example, the circuitry may orient the data model of the fetus accordingly and generate the visualization based on the oriented data model.

In some embodiments, the circuitry is further configured to determine a position of a display device relative to the mother; and the generating of the visualization of the fetus includes generating the visualization of the fetus in a perspective as seen from the position of the display device.

The image data of the belly of the mother may be acquired with a camera of a mobile device (e.g., smartphone, tablet, notebook, head-mounted display, smartglasses, smartwatch, AR/VR device, etc.). A user may direct the camera of the mobile device to the belly of the mother. The mobile device may further include a display device (e.g., liquid crystal display (LCD), organic light emitting diode (OLED), quantum dot display (QLED), plasma display panel (PDP), cathode-ray tube (CRT) or the like), the circuitry may send the generated visualization of the fetus to the mobile device, and the mobile device may cause its display device to display the visualization of the fetus.

A positional relationship between the camera of the mobile device and the display device of the mobile device may be predefined (e.g., the camera may be oriented in an opposite direction than the display device, without limiting the disclosure to such a configuration). The circuitry may then determine that the perspective as seen from the position of the display device corresponds to a perspective as seen by the camera. The circuitry may determine the position of the display device relative to the mother (e.g., relative to the belly of the mother or to a portion of the belly that is represented in the image data) based on the image data (e.g., based on a perspective from which the belly is represented in the image data).

When a user moves the mobile device with the camera relative to the belly of the mother, the circuitry may update the visualization of the fetus (e.g., a perspective from which the fetus is visualized) according to the changed position of the camera.

For example, the fetus may be anchored in relation to the belly of the mother using the mobile device (e.g., AR device, smartphone, smartglasses, etc.). For example, a user may point the mobile (portable/wearable) device for visualization directly at the belly of the mother to provide a real-time look “inside” the womb. This way, an exact scale and pose may be precisely visualized, and may thus give a real insight and feeling of a state of the fetus/baby. Accordingly, a visualization of the fetus using an AR device to “look through” into the belly of the mother through a physical barrier and better understand a real-time position and movements of the fetus/baby may be possible.

The image data may represent a greyscale image, a colored (e.g., red-green-blue (RGB)) image, a depth image (e.g., obtained based on time-of-flight (ToF) imaging), an infrared image, or the like.

However, the disclosure is not limited to obtaining the image data with a mobile device that displays the visualization of the fetus. For example, the camera may be provided separately from the mobile device and/or display device, and a positional relation between the display device and the camera may be determined based on, e.g., sound ranging, radio ranging and/or identifying the display device in the image data.

In some embodiments, the generating of the visualization of the fetus includes reconstructing a missing portion of the fetus, the missing portion being a portion that is not indicated by the ultrasound data.

The missing portion of the fetus may include a portion of the fetus that could not be captured in the ultrasound data because of an orientation and/or posture of the fetus (e.g., the fetus may “hide” a body part from ultrasound capturing), because of a position of the ultrasound probe (e.g., a body part of the fetus may be outside of a field-of-view of the ultrasound probe), and/or because of a penetration depth of the ultrasound signal (e.g., the ultrasound signal may not reach a body part (e.g., an organ or a bone) of the fetus with a sufficient signal strength).

The generative AI may be trained, based on a typical anatomy of a fetus, to reconstruct the missing portion of the fetus, such that a visualization of all body parts of the fetus and/or from any perspective may be possible.

Accordingly, if some parts of the fetus could not be captured due to a position of the fetus inside the womb and/or ultrasound probe positioning, such parts may be automatically generated by the generative AI using generative AI methods such as NeRF or similar.

The circuitry may indicate reconstructed parts of the fetus as generated instead of directly captured in order to ensure a quality of the visualization, for example, such that a doctor may recognize from the visualization that a body part of the fetus has been reconstructed by the generative AI.

The circuitry may indicate the reconstructed parts as generated based on a color (e.g., the reconstructed parts may be visualized in another color than observed parts of the fetus), and/or based on a frame, a label, a hatching or the like added to the visualization. A user may switch between a raw mode and an augmented mode. The raw mode may be a mode in which the reconstructed parts are indicated as generated or in which reconstructing of parts of the fetus is deactivated (e.g., for a doctor who is examining the fetus), and the augmented mode may be a mode in which the missing parts are reconstructed without an indication which parts are reconstructed (e.g., for parents of the fetus who want to see an image of their baby).

In some embodiments, the missing portion includes an organ of the fetus. Generally, in some embodiments, the missing portion includes external and/or internal features of the fetus such as skin, hair, eyes, bones, muscles, organs, etc.

The generative Al may be used to extend an outlook further to reconstruct a real view of the fetus, including a realistic rendering of the external and internal features such as skin, hair, eyes, bones, muscles, and organs of the fetus. Such information may be useful not only for expecting parents to “meet” their baby, but may also give more precise information for medical professionals to investigate a health and state of the fetus/baby, and make more precise measurements.

In some embodiments, the ultrasound data represent the fetus at two different dates; and the circuitry is configured to interpolate the fetus for a date between the two different dates.

The interpolating of the fetus may include interpolating a state of the fetus, e.g., an appearance, a size, a posture, an orientation, a developmental stage etc. of the fetus. The interpolating may be performed by using the generative AI. The circuitry may generate a visualization of the interpolated (state of the) fetus as a video, as a sequence of images or the like. For example, the video or sequence of images, respectively, may visualize a change of the state of the fetus as a smooth transition and/or as a sequence of developmental steps. The two different dates may correspond to dates at which an ultrasound examination of the fetus is performed.

For example, in subsequent visits at a doctor, a growth “timeline” of the fetus/baby may be created, and a development of the fetus may be followed precisely over time.

Accordingly, based on multiple ultrasound scans (and resulting ultrasound data; e.g. ultrasound data from different dates) and the data model of the fetus/baby in the womb, a growth of the baby may be interpolated for uncaptured days in-between the ultrasound scans. By incorporating data from all the ultrasound scans, a full development cycle may be interpolated (and possibly extrapolated as necessary) to show the growth timeline and for capturing features of the specific fetus/baby. An arbitrary number of ultrasound scans may be used for generating the visualization of the fetus according to the growth timeline.

An outcome of generating the growth timeline may be specific to the observed fetus/baby, and may showcase an exact captured anatomy of the fetus during the ultrasound scans and an interpolation of in-between steps using the generative AI. The visualization of the growth timeline may be possible from any angle. Both, doctors and parents may benefit from observing the development of the fetus/baby in detail.

In some embodiments, the data model includes a three-dimensional (3D) representation of the fetus. The 3D representation of the fetus in the data model may allow for generating the visualization of the fetus from any direction.

In some embodiments, the ultrasound data represent the fetus in respective perspectives as seen from multiple directions.

For example, the visualization of the fetus may be based on ultrasound data from different ultrasound measurements, e.g., based on ultrasound data acquired from different positions of the belly of the mother. Thus, the data model (and the visualization) of the fetus may indicate features of the fetus that cannot be observed simultaneously within a single ultrasound measurement.

In some embodiments, the circuitry is further configured to generate an instruction for positioning an ultrasound sensor at the belly of the mother.

The circuitry may cause the instruction to be output to a user (e.g., a doctor) who is performing an ultrasound examination of the fetus. The instruction may indicate a direction in which the ultrasound probe should be moved or turned such that a feature of the fetus can be observed that is not represented in previously obtained ultrasound data.

The instruction may include an acoustic message (e.g., a speech output and/or a beep sound) and may be output by a loudspeaker provided at and/or associated with the ultrasound probe, for example. The instruction may include a visible message (e.g., a text message, an arrow, a light-emitting diode (LED), or the like) and may be output by the ultrasound probe and/or by the display device that displays the visualization, for example.

Furthermore, during capturing the ultrasound data, a positioning of the ultrasound probe may be guided by the circuitry to estimate how it should be held to capture “unseen” parts in the current ultrasound scan. This may enable not only medical professionals, but casual users, such as expecting parents or their relatives to perform an ultrasound scan at home.

The circuitry may determine a current position of the ultrasound probe based on the ultrasound data, for example, based on a perspective in which the fetus is represented in the ultrasound data, based on structures of the body of the mother that are represented in the ultrasound data (e.g., a bone (e.g., hip, vertebra, rib, etc.), a tissue, a muscle, an organ, a blood vessel, the uterus or the like), and/or based on a representation of the ultrasound probe in image data of the belly of the mother. The circuitry may use the determined position of the ultrasound probe for guidance to get a precise relative position between the ultrasound probe (and/or a mobile device, as described above) and the baby/fetus in the womb.

Some embodiments pertain to a method for generating a visualization of a fetus, wherein the method includes: obtaining ultrasound data representing a fetus in a uterus of its mother; establishing, based on the ultrasound data, a data model of the fetus; and generating, based on the data model, a visualization of the fetus using a generative artificial intelligence.

The method may be configured similar to the circuitry described above, and any feature described with respect to the circuitry may correspond to a respective feature of the method. The features described above with respect to the circuitry and/or method may be combined in any suitable way.

The methods as described herein are also implemented in some embodiments as a computer program causing a computer and/or a processor to perform the method, when being carried out on the computer and/or processor. In some embodiments, also a non-transitory computer-readable recording medium is provided that stores therein a computer program product, which, when executed by a processor, such as the processor described above, causes the methods described herein to be performed.

Returning to FIG. 1, FIG. 1 illustrates an embodiment of a circuitry 1. The circuitry 1 includes a processing unit 2, an artificial intelligence (AI) processing unit 3, a memory unit 4 and a communication unit 5. The processing unit 2, the AI processing unit 3, the memory unit 4 and the communication unit 5 can communicate with each other via a bus.

The processing unit 2 includes a central processing unit (CPU) and is configured to perform general information processing and control the AI processing unit 3, the memory unit 4 and the communication unit 5.

The AI processing unit 3 includes a graphics processing unit (GPU) and is configured to execute a generative AI 3a. The generative AI 3a is based on a neural radiance field (NeRF) and is configured to generate a 3D visualization of a fetus 12 based on a 3D data model of the fetus 12 and on ultrasound data of the fetus.

The memory unit 4 includes a dynamic random-access memory (DRAM) and a solid-state drive (SSD), and is configured to store instructions for the processing unit 2, parameters of the generative AI 3a, ultrasound data obtained by the circuitry 1, a data model of the fetus 12 established by the circuitry 1, a visualization of the fetus 12 generated by the generative AI 3a, and further data that are necessary for a function of the circuitry 1.

A belly 10 of a mother includes a uterus 11, and the uterus 11 includes the fetus 12. An ultrasound probe 13 acquires ultrasound data of the fetus 12 in the uterus 11 and transmits the ultrasound data to the communication unit 5 of the circuitry 1 via a USB connection 13a.

The processing unit 2 obtains the ultrasound data from the ultrasound probe 13 that represent the fetus 12 in the uterus 11, and establishes a data model of the fetus 12 based on the ultrasound data. The AI processing unit 3 then generates, by using the generative AI 3a, a visualization of the fetus 12 based on the data model.

The communication unit 5 sends the generated visualization of the fetus 12 to a screen 14 (an example of a display unit) via a high-definition multimedia interface (HDMI) connection 14a. The screen 14 is mounted in a surgery of a doctor who performs an ultrasound examination of the fetus 12. The screen 14 displays the visualization of the fetus 12 such that the doctor can examine the fetus 12.

A smartphone 15 (an example of a mobile device) acquires, with a camera provided in the smartphone 15, image data that represent the belly 10 of the mother and transmits the image data via a Wi-Fi connection 15a to the communication unit 5 of the circuitry 1.

The AI processing unit 3 generates a visualization of the fetus 12 in which the fetus 12 is shown from a perspective of the smartphone 15 (and its camera) in an orientation that corresponds to an orientation of the belly 10 in the image data, and wherein an image of the fetus 12 is inserted into an image of the belly 10 represented by the image data.

The communication unit 5 sends the visualization via the Wi-Fi connection 15a to the smartphone 15, and the smartphone 15 causes a display (an example of a display unit) of the smartphone 15 to display the visualization, such that parents (i.e., the mother and a father) of the fetus 12 can look at the fetus 12 on the smartphone 15 as if they were “looking through” the belly 10 of the mother.

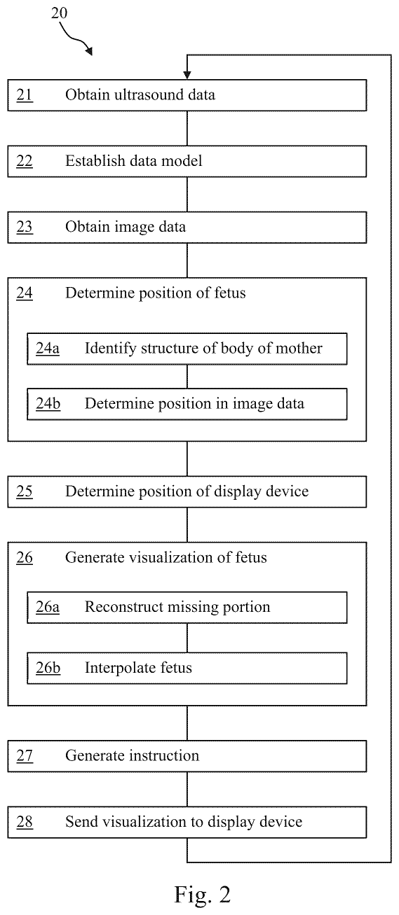

The circuitry 1 and its units 2 to 5 are configured to perform the method described with respect to FIG. 2.

FIG. 2 illustrates an embodiment of a method 20. The method 20 of FIG. 2 is an example of a method executed by the circuitry 1 of FIG. 1 and its units 2 to 5.

At 21 of the method 20, the processing unit 2 obtains, via the communication unit 5 from the ultrasound probe 13, ultrasound data that represent the fetus 12 in the uterus 11 of its mother. The ultrasound data represent the fetus 12 in respective perspectives as seen from multiple directions.

At 22 of the method 20, the processing unit 2 establishes, based on the ultrasound data obtained at 21, a data model of the fetus 12. The data model includes a 3D representation of the fetus 12.

At 23 of the method 20, the processing unit 2 obtains, via the communication unit 5 from the smartphone 15, image data of a portion of the belly 10 of the mother.

At 24 of the method 20, the processing unit 2 determines a position of the fetus 12 relative to the mother (i.e., relative to the belly 10 of the mother). The determining of the position of the fetus 12 at 24 includes identifying, at 24a, in the ultrasound data, a structure of a body of the mother, and determining, at 24b, in an image represented by the image data, a position that corresponds to the structure.

At 25 of the method 20, the processing unit 2 determines a position of the smartphone 15 (and, thus, of the display of the smartphone 15, which is an example of a display device) relative to the mother (i.e., relative to the belly 10) based on the image data (i.e., based on a perspective from which the belly 10 is represented in the image data).

At 26 of the method 20, the AI processing unit 3 generates, based on the data model established at 22, based on the ultrasound data obtained at 21, and based on the image data obtained at 23, a visualization of the fetus 12 using the generative AI 3a, wherein the generating of the visualization includes executing the generative AI 3a. The AI processing unit 3 generates the visualization according to the determined position of the fetus 12 that has been determined at 24 and according to the determined position of the display device 15 that has been determined at 25, in a perspective as seen from the position of the display device 15.

The generating of the visualization of the fetus 12 at 26 includes reconstructing, at 26a, a missing portion of the fetus 12, wherein the missing portion is a portion of the fetus 12 that is not indicated by the ultrasound data. The missing portion includes an organ of the fetus 12 (without limiting the disclosure to reconstructing an organ; the missing portion may in some embodiments include any external and/or internal feature of the fetus 12 such as skin, hair, eyes, bones, muscles, organs, etc.).

The generating of the visualization of the fetus 12 at 26 includes interpolating, at 26b, using the generative AI 3a, (a state of) the fetus 12 for a date between two different dates, wherein the two different dates correspond to dates at which an ultrasound examination of the fetus 12 is performed, and the ultrasound data represent the (state of the) fetus 12 at the two different dates. The interpolating at 26b includes generating a video in which a smooth transition between the state of the fetus 12 at the two different dates is shown, such that a growth timeline of the fetus 12 is visualized.

At 27 of the method 20, the processing unit 2 generates an instruction for positioning an ultrasound sensor of the ultrasound probe 13 at the belly 10 of the mother. The instruction indicates a position to which the ultrasound probe 13 (with the ultrasound sensor) should be moved in order to acquire ultrasound data of a portion of the fetus 12 that is not yet represented in the ultrasound data in sufficient detail. The processing unit 2 causes both the screen 14 and a light-emitting diode (LED)-based display of the ultrasound probe 13 to display an arrow that corresponds to a direction in which the ultrasound probe 13 should be moved.

At 28, the communication unit 5 sends the visualization of the fetus 12 that has been generated at 26 to a display device. The sending at 28 includes sending the visualization to the screen 14 such that a doctor who is performing an ultrasound examination of the fetus 12 can examine the fetus 12. The sending at 28 also includes sending the visualization to the smartphone 15 such that parents of the fetus 12 can view the fetus 12. The sending at 28 also includes sending, to the smartphone 15, the video generated at 26b that visualizes the growth timeline of the fetus 12. The smartphone 15 stores the video such that the parents of the fetus 12 can watch the video again at a later time.

As indicated in FIG. 2 by the arrow from 28 to 21, the circuitry 1 executes a next run of the method 20 such that the fetus 12 can be visualized in real time (e.g., at a frame rate of 30 fps, without limiting the disclosure to this value). In the next run of the method 20, the processing unit 2 obtains more recent ultrasound data at 21, updates the data model at 22, obtains more recent image data at 23, determines an updated position of the fetus 12 at 24, determines an updated position of the display device 15 at 25, generates an updated visualization of the fetus 12 at 26, generates an updated instruction for positioning of the ultrasound probe 13 at 27, and sends the updated visualization of the fetus 12 to the display device 14 and 15 at 28. The circuitry 1 executes a next run of the method 20 during an ongoing ultrasound examination and stops executing a next run of the method 20 when a user stops the ultrasound examination.

Note that, in some embodiments, one or more parts of the method 20 of FIG. 2 are omitted. In some embodiments, the determining of the position of the display device 15 at 25 is omitted, and the visualization is generated at 26 independent of a position of the display device 15 relative to the belly 10 of the mother. In addition, in some embodiments, the obtaining of the image data at 23 and the determining of the position of the fetus at 24 is also omitted, and the visualization is generated at 26 independent of a position of the fetus 12 relative to (the belly 10 of) the mother. In some embodiments, the reconstructing of a missing portion of the fetus 12 at 26a is omitted. In some embodiments, the interpolating of (the state of) the fetus 12 at 26b is omitted. In some embodiments, the generating of the instruction at 27 is omitted. The skilled person will find further suitable variations of the method 20 in which one or more parts of the method 20 may be omitted and/or in which an ordering of parts of the method 20 may be changed.

FIG. 3 illustrates an embodiment of a general-purpose computer 150. The general-purpose computer 150 is an example of an information processing apparatus that includes circuitry that is configured to perform the method according to the present technology (e.g., the method 20 of FIG. 2). The computer 150 has components 151 to 161, which can form a circuitry, such as the circuitry 1 of FIG. 1, as described herein.

Embodiments which use software, firmware, programs or the like for performing the methods as described herein can be installed on computer 150, which is then configured to be suitable for the concrete embodiment.

The computer 150 has a CPU 151 (Central Processing Unit), which can execute various types of procedures and methods as described herein, for example, in accordance with programs stored in a read-only memory (ROM) 152, stored in a storage 157 and loaded into a random-access memory (RAM) 153, stored on a medium 160 which can be inserted in a respective drive 159, etc.

Furthermore, the computer 150 includes an artificial intelligence (AI) processor 151a. The AI processor 151a may include a graphics processing unit (GPU) and/or a tensor processing unit (TPU). The AI processor 151a may be configured to execute an AI model (e.g., an artificial neural network), for example, the generative AI 3a of FIG. 1.

The CPU 151, the ROM 152 and the RAM 153 are connected with a bus 161, which in turn is connected to an input/output interface 154. The number of CPUs, memories and storages is only exemplary, and the skilled person will appreciate that the computer 150 can be adapted and configured accordingly for meeting specific requirements which arise, when it functions as a base station or as user equipment (end terminal).

At the input/output interface 154, several components are connected: an input 155, an output 156, the storage 157, a communication interface 158 and the drive 159, into which a medium 160 (compact disc (CD), digital video disc (DVD), universal serial bus (USB) flash drive, secure digital (SD) card, CompactFlash (CF) memory, or the like) can be inserted.

The input 155 can be a pointer device (mouse, graphic table, or the like), a keyboard, a microphone, a camera, a touchscreen, an eye-tracking unit etc.

The output 156 can have a display (liquid crystal display (LCD), cathode ray tube (CRT) display, light-emitting diode (LED) display, electronic paper, etc.; e.g., included in a touchscreen), loudspeakers, etc.

The storage 157 can have a hard disk drive (HDD), a solid-state drive (SSD), a flash drive and the like.

The communication interface 158 can be adapted to communicate, for example, via universal serial bus (USB), a serial port (RS-232), parallel port (IEEE 1284), a local area network (LAN; e.g., ethernet), wireless local area network (WLAN; e.g., Wi-Fi, IEEE 802.11), mobile telecommunications system (GSM, UMTS, LTE, NR, etc.), Bluetooth, near-field communication (NFC), ZigBee, infrared, etc.

It should be noted that the description above only pertains to an example configuration of computer 150. Alternative configurations may be implemented with additional or other sensors, storage devices, interfaces or the like. For example, the communication interface 158 may support other radio access technologies than the mentioned UMTS, LTE and NR.

It should be recognized that the embodiments describe methods with an exemplary ordering of method steps. The specific ordering of method steps is however given for illustrative purposes only and should not be construed as binding. For example, the ordering of 21 and 22 in the embodiment of FIG. 2 may be exchanged. Also, the ordering of 24 and 25 in the embodiment of FIG. 2 may be exchanged. Further, 23, 24 and/or 25 may be performed before 22 or 21 in the embodiment of FIG. 2. Further, also the ordering of 26 and 27 in the embodiment of FIG. 2 may be exchanged. Other changes of the ordering of method steps may be apparent to the skilled person.

Please note that the division of the circuitry 1 into units 2 to 5 is only made for illustration purposes and that the present disclosure is not limited to any specific division of functions in specific units. For instance, the circuitry 1 could be implemented by a respective programmed processor, field programmable gate array (FPGA) and the like.

The method of FIG. 2 can also be implemented as a computer program causing a computer and/or a processor, such as the circuitry 1 discussed above, to perform the method, when being carried out on the computer and/or processor. In some embodiments, also a non-transitory computer-readable recording medium is provided that stores therein a computer program product, which, when executed by a processor, such as the processor described above, causes the method described to be performed.

All units and entities described in this specification and claimed in the appended claims can, if not stated otherwise, be implemented as integrated circuit logic, for example on a chip, and functionality provided by such units and entities can, if not stated otherwise, be implemented by software.

In so far as the embodiments of the disclosure described above are implemented, at least in part, using software-controlled data processing apparatus, it will be appreciated that a computer program providing such software control and a transmission, storage or other medium by which such a computer program is provided are envisaged as aspects of the present disclosure.

Note that the present technology can also be configured as described below.
(1) A circuity for generating a visualization of a fetus, the circuitry being configured to:
obtain ultrasound data representing a fetus in a uterus of its mother;
establish, based on the ultrasound data, a data model of the fetus; and
generate, based on the data model, a visualization of the fetus using a generative artificial intelligence.
(2) The circuitry of (1),
wherein the circuitry is further configured to:
   obtain image data of a portion of a belly of the mother; and
   determine a position of the fetus relative to the mother; and
wherein the generating of the visualization of the fetus includes generating the visualization according to the determined position.
(3) The circuitry of (2), wherein the determining of the position of the fetus includes:
identifying, in the ultrasound data, a structure of a body of the mother; and
determining, in an image represented by the image data, a position that corresponds to the structure.
(4) The circuitry of (2) or (3),
wherein the circuitry is further configured to determine a position of a display device relative to the mother; and
wherein the generating of the visualization of the fetus includes generating the visualization of the fetus in a perspective as seen from the position of the display device.
(5) The circuitry of any one of (1) to (4),
wherein the generating of the visualization of the fetus includes reconstructing a missing portion of the fetus, the missing portion being a portion that is not indicated by the ultrasound data.
(6) The circuitry of (5),
wherein the missing portion includes an organ of the fetus.
(7) The circuitry of any one of (1) to (6),
wherein the ultrasound data represent the fetus at two different dates; and
wherein the circuitry is configured to interpolate the fetus for a date between the two different dates.
(8) The circuitry of any one of (1) to (7),
wherein the data model includes a three-dimensional representation of the fetus.

(9) The circuitry of any one of (1) to (8),
wherein the ultrasound data represent the fetus in respective perspectives as seen from multiple directions.
(10) The circuitry of any one of (1) to (9),
wherein the circuitry is further configured to generate an instruction for positioning an ultrasound sensor at the belly of the mother.
(11) A method for generating a visualization of a fetus, the method comprising:
obtaining ultrasound data representing a fetus in a uterus of its mother;
establishing, based on the ultrasound data, a data model of the fetus; and
generating, based on the data model, a visualization of the fetus using a generative artificial intelligence.
(12) The method of (11),
wherein the method further comprises:
   obtaining image data of a portion of a belly of the mother; and
   determining a position of the fetus relative to the mother; and
wherein the generating of the visualization of the fetus includes generating the visualization according to the determined position.
(13) The method of (12), wherein the determining of the position of the fetus includes:
identifying, in the ultrasound data, a structure of a body of the mother; and
determining, in an image represented by the image data, a position that corresponds to the structure.
(14) The method of (12) or (13),
wherein the method further comprises determining a position of a display device relative to the mother; and
wherein the generating of the visualization of the fetus includes generating the visualization of the fetus in a perspective as seen from the position of the display device.
(15) The method of any one of (11) to (14),
wherein the generating of the visualization of the fetus includes reconstructing a missing portion of the fetus, the missing portion being a portion that is not indicated by the ultrasound data.
(16) The method of (15),
wherein the missing portion includes an organ of the fetus.
(17) The method of any one of (11) to (16),
wherein the ultrasound data represent the fetus at two different dates; and
wherein the method comprises interpolating the fetus for a date between the two different dates.
(18) The method of any one of (11) to (17),
wherein the data model includes a three-dimensional representation of the fetus.
(19) The method of any one of (11) to (18),
wherein the ultrasound data represent the fetus in respective perspectives as seen from multiple directions.
(20) The method of any one of (11) to (19),
wherein the method further comprises generating an instruction for positioning an ultrasound sensor at the belly of the mother.
(21) A computer program comprising program code causing a computer to perform the method according to anyone of (11) to (20), when being carried out on a computer.
(22) A non-transitory computer-readable recording medium that stores therein a computer program product, which, when executed by a processor, causes the method according to anyone of (11) to (20) to be performed.

1. A circuity for generating a visualization of a fetus, the circuitry being configured to:
obtain ultrasound data representing a fetus in a uterus of its mother;
establish, based on the ultrasound data, a data model of the fetus; and
generate, based on the data model, a visualization of the fetus using a generative artificial intelligence.

2. The circuitry of claim 1,
wherein the circuitry is further configured to:
obtain image data of a portion of a belly of the mother; and
determine a position of the fetus relative to the mother; and
wherein the generating of the visualization of the fetus includes generating the visualization according to the determined position.

3. The circuitry of claim 2, wherein the determining of the position of the fetus includes:
identifying, in the ultrasound data, a structure of a body of the mother; and
determining, in an image represented by the image data, a position that corresponds to the structure.

4. The circuitry of claim 2,
wherein the circuitry is further configured to determine a position of a display device relative to the mother; and
wherein the generating of the visualization of the fetus includes generating the visualization of the fetus in a perspective as seen from the position of the display device.

5. The circuitry of claim 1,
wherein the generating of the visualization of the fetus includes reconstructing a missing portion of the fetus, the missing portion being a portion that is not indicated by the ultrasound data.

6. The circuitry of claim 5,
wherein the missing portion includes an organ of the fetus.

7. The circuitry of claim 1,
wherein the ultrasound data represent the fetus at two different dates; and
wherein the circuitry is configured to interpolate the fetus for a date between the two different dates.

8. The circuitry of claim 1,
wherein the data model includes a three-dimensional representation of the fetus.

9. The circuitry of claim 1,
wherein the ultrasound data represent the fetus in respective perspectives as seen from multiple directions.

10. The circuitry of claim 1,
wherein the circuitry is further configured to generate an instruction for positioning an ultrasound sensor at the belly of the mother.

11. A method for generating a visualization of a fetus, the method comprising:
obtaining ultrasound data representing a fetus in a uterus of its mother;
establishing, based on the ultrasound data, a data model of the fetus; and
generating, based on the data model, a visualization of the fetus using a generative artificial intelligence.

12. The method of claim 11,
wherein the method further comprises:
obtaining image data of a portion of a belly of the mother; and
determining a position of the fetus relative to the mother; and
wherein the generating of the visualization of the fetus includes generating the visualization according to the determined position.

13. The method of claim 12, wherein the determining of the position of the fetus includes:
identifying, in the ultrasound data, a structure of a body of the mother; and
determining, in an image represented by the image data, a position that corresponds to the structure.

14. The method of claim 12,
wherein the method further comprises determining a position of a display device relative to the mother; and
wherein the generating of the visualization of the fetus includes generating the visualization of the fetus in a perspective as seen from the position of the display device.

15. The method of claim 11,
wherein the generating of the visualization of the fetus includes reconstructing a missing portion of the fetus, the missing portion being a portion that is not indicated by the ultrasound data.

16. The method of claim 15,
wherein the missing portion includes an organ of the fetus.

17. The method of claim 11,
wherein the ultrasound data represent the fetus at two different dates; and
wherein the method comprises interpolating the fetus for a date between the two different dates.

18. The method of claim 11,
wherein the data model includes a three-dimensional representation of the fetus.

19. The method of claim 11,
wherein the ultrasound data represent the fetus in respective perspectives as seen from multiple directions.

20. The method of claim 11,
wherein the method further comprises generating an instruction for positioning an ultrasound sensor at the belly of the mother.
